(19) United States
(12) Patent Application Publication
(10) Pub. No.: US 2023/0240689 A1
(43) Pub. Date: Aug. 3, 2023

(54) AUTOMATIC UNLOCK DEVICE FOR TOURNIQUET

(71) Applicant: INGENIO TECHNOLOGY CO., LIMITED, Chai Wan (HK)

(72) Inventors: Man Yin Arthur Newton CHU, Chai Wan (HK); Kwok Chor Dickson TAM, Chai Wan (HK); Fu Keung Derek YAU, Chai Wan (HK)

(21) Appl. No.: 17/587,808

(22) Filed: Jan. 28, 2022

(51) Int. Cl. A61B 17/132

(52) U.S. Cl. CPC A61B 17/1327; A61B 2017/00398

(57) ABSTRACT

A automatic unlock device for tourniquet, includes a lock structure for setting the tourniquet in tight state, an unlock device for unlocking the lock structure and setting the tourniquet in loose state, and a control circuit for controlling the unlock device. The unlock device is connected with the lock structure, the drive part of the unlock device is connected with the control circuit, and the drive part drives the unlock device to lock or loose the lock structure. When in use, the lock structure is buckled with the unlock device to reduce the length of the tourniquet, that is, in tight state. Simultaneously, the control circuit can be manually or automatically set the unlocking time and start timing. When the time is up, the control circuit outputs a control signal to the drive part to drive the unlock device releasing the lock structure. Then, the tourniquet is in loose state, thereby to prevent medical accidents, such as ischemia, necrosis of the distal limbs, which is caused by forgetting or accidentally using the tourniquet for a long time.

### FIELD OF THE INVENTION

The present invention involves a structure of a tourniquet, especially an automatic unlock device for controlling the tourniquet.

### BACKGROUND OF THE INVENTION

The tourniquet is usually used during routine treatment, infusion, blood drawing, blood transfusion, and hemostasis for just one-time use. It compressed blood vessels to block blood flow and achieve hemostasis. However, if improper used or lasted too long, the tourniquet may cause ischemia, necrosis of the distal limbs and disability even. Especially in the infusion or blood drawing, the operation must be performed after the binding of the tourniquet. But when the operation is completed, it is forgotten to remove tourniquet in some reasons, and that may cause the above accidents.

### SUMMARY OF THE UTILITY MODEL

In view of the aforesaid drawbacks of the prior art, the present utility model provides an automatic unlock device for tourniquet, to realize the tightening of the tourniquet when the device under use, and automatically unlock to loosen the tourniquet after operation to prevent the ischemia of the distal limbs caused by forgetting or accidentally using the tourniquet for a long time which may cause necrosis and other medical accidents.

So, the present invention provides:

Automatic unlock device for tourniquet, wherein includes a lock structure for setting the tourniquet in tight state, an unlock device for unlocking and setting the tourniquet in loose state, and a control circuit for controlling the unlock device; the unlock device is connected to the lock structure, the drive part of the unlock device connects to the control circuit, and the drive part drives the unlock device to lock to or loose from the lock structure.

Further, the lock structure is a lock buckle arranged in the tourniquet; while locked, the lock buckle is buckled with a lock body in the unlock device to set the tourniquet in tight state; while unlocked, the lock buckle loose from the lock body to set the tourniquet in loose state; the lock body connects to the drive part of the unlock device.

Further, the lock structure is a crimping structure provided on the unlock device, which includes a crimping cover that is configured to relatively open/close with the crimping surface of the unlock device; a rib is provided on the crimping surface or the inner surface of the crimping cover; while locked, the tourniquet is folded along the longitudinal direction and placed in the crimping cover; the crimping cover and the crimping surface are closed to let the rib press against the folded part of the tourniquet, so the tourniquet is set in tight state; while unlocked, the crimping cover and the crimping surface are opened to let the rib loosen the folded part of the tourniquet, and the tourniquet is set in loose state.

Further, a groove is provided at a position opposite to the rib, and the rib presses the folded part of the tourniquet into the groove to fix.

Further, one end of the crimping cover is connected with the unlock device through a shaft, and the other end is buckled with the lock body of the unlock device by the lock buckle.

Further, the lock structure and the unlock device are winding structures, which include a winding shaft for the tourniquet being connected with; and the winding shaft is connected with the drive part; while locked, Further, t the drive part drives the winding shaft to wind the tourniquet on it, so the tourniquet is set in tight state; while unlocked, the drive part drives the winding shaft in the reverse direction or disconnects with the winding shaft to release the tourniquet for setting it in loose state.

Further, a lock hole and a bolt are respectively provided with the lock buckle and the lock body, and the bolt is locked by the lock hole after being inserted into the lock hole; the movement of the lock buckle and the lock body intersects.

Further, the lock body is provided with a unlock button, the unlock button is connected to the lock hole, and the unlock button is pressed to move the lock hole to unlock the bolt.

Further, the unlock device is provided with an ejection structure along the insertion direction of the lock buckle, which at least includes a spring; the lock buckle compresses the spring when inserted, and the spring ejects the lock buckle when unlocked.

Further, the drive part is a solenoid, and the end of the lock body is matched with the movable part of the solenoid.

Further, the drive part is a motor, the shaft of the motor is provided with an eccentric wheel, and the end of the lock body is matched with the eccentric wheel.

Further, a activate button for setting the timing of unlocking is provided, and the activate button is connected to the control circuit.

Further, the activate button is arranged on the surface of the unlock device where is contact with the body.

Further, an alarm device is provided in the control circuit.

The utility model has the following advantages: When in use, the lock structure is buckled with the unlock device to reduce the length of the tourniquet, that is, the tourniquet is in tight state. At the same time, the control circuit will manually or automatically set the unlock time and start counting time. When the timing is over, the control circuit outputs a control signal to the drive part, and the drive part drives the unlock device to release the lock structure. Then, the tourniquet is loosened, thereby to prevent medical accidents, such as ischemia, necrosis of the distal limbs, which is caused by forgetting or accidentally using the tourniquet for a long time.

### BRIEF DESCRIPTION OF THE DRAWINGS

Below are explanations of the invention combining with drawings.

FIG. 1 is the front view of the first embodiment of the present invention.

FIG. 2 is the diagram of the internal structure of the first embodiment.

FIG. 3 is the perspective view of the second embodiment of the present invention.

FIG. 4 is the explosive view of FIG. 3.

FIG. 5 is the perspective view from other direction of the second embodiment of the present invention.

In the figures: 1. the tourniquet, 2. the lock structure, 3. the unlock device, 4. the lock buckle, 5. the lock body, 6. the crimping cover, 7. the rib, 8. the groove, 9. the unlock button, 10. the ejection structure, 11. the activate button.

### DESCRIPTION OF THE EMBODIMENTS

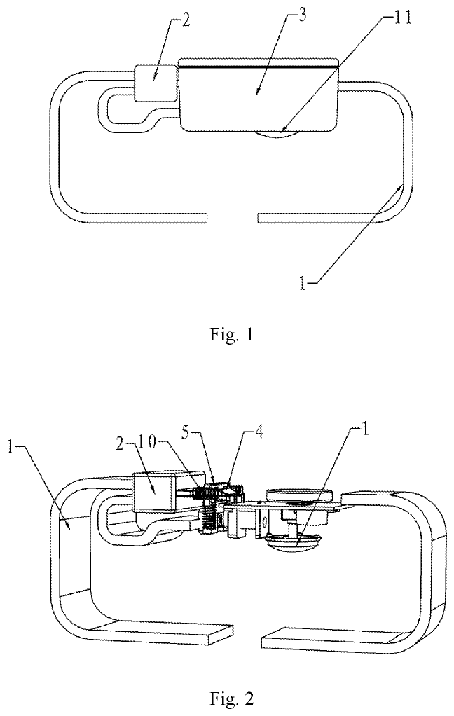

Refer to FIG. 1, the automatic tourniquet unlock device includes the lock structure 2 for setting the tourniquet 1 in tight state, the unlock device 3 for unlocking the lock structure 2 and setting the tourniquet 1 in loose state, and a control circuit for controlling the unlock device 3. The unlock device 3 is connected to the lock structure 2. The drive part of the unlock device 3 is electrically connected to the control circuit. The drive part drives the unlock device 3 to lock or release the lock structure 2. When in use, the lock structure 2 is buckled with the unlock device 3 to reduce the length of the tourniquet, that is, the tourniquet is in tight state. Then, the control circuit will manually or automatically set the unlock time and start counting time. When the timing is over, the control circuit outputs a control signal to the drive part, and the drive part drives the unlock device 3 to release the lock structure 2. Then, the tourniquet 1 is loosened, thereby to prevent medical accidents, such as ischemia, necrosis of the distal limbs, which is caused by forgetting or accidentally using the tourniquet for a long time.

The above-mentioned lock structure 2 can facilitate in various structures to realize the tightening and loosening of the tourniquet 1. Below, the present invention shows some embodiments for further explanation and description.

### Embodiment 1

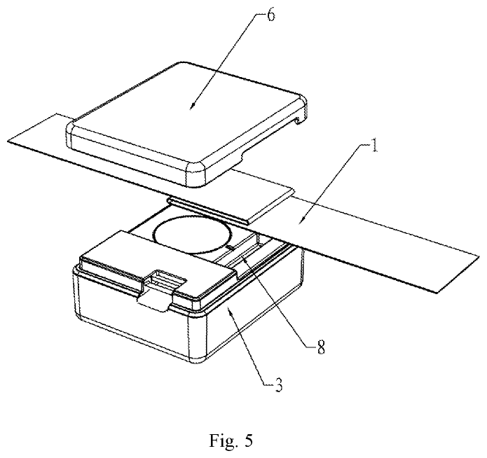

Refer to FIG. 2 to FIG. 5, the lock structure 2 is a lock buckle 4 arranged in the tourniquet 1, and the lock body 5 is provided in the unlock device 3. The lock body 5 is connected to the drive part of the unlock device 3 to drive the lock body 5 to move and realize the locking and unlocking of the lock buckle 4.

When locked, the lock buckle 4 is buckled with the lock body 5 in the unlock device 3. Since the lock buckle 4 is arranged in the middle of the tourniquet 1, the circumference of the tourniquet 1 loop is shortened, so let the tourniquet 1 be set in tight state.

When unlocked, the lock buckle 4 loose from the lock body 5, and the circumference of the tourniquet 1 loop becomes longer, so let the tourniquet 1 be set in loose state.

### Embodiment 2

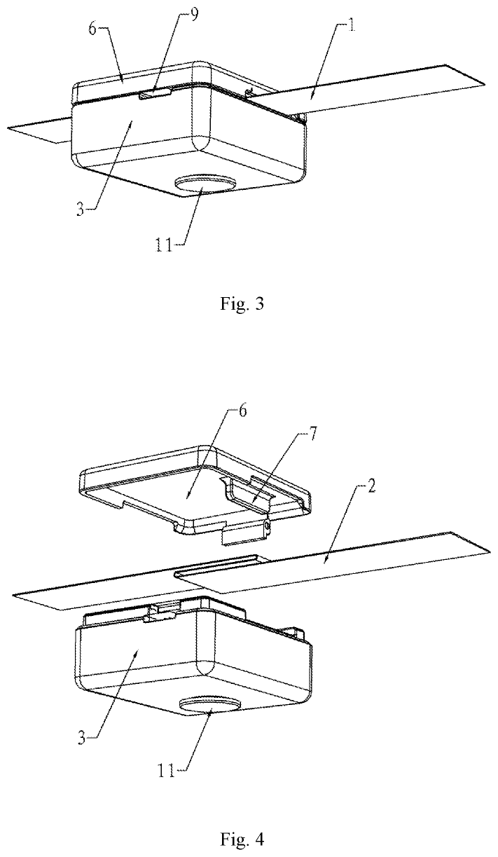

Refer to FIG. 3 to FIG. 5, the lock structure 2 is a crimping structure provided on the unlock device 3. It includes a crimping cover 6. The crimping cover 6 and the crimping surface of the lock structure 2 can make movement between, so that to facilitate pressing and loosening the tourniquet 1. At the same time, in order to compress the tourniquet 1 while tightening, the crimping surface or the inner surface of the crimping cover 6 is provided with the rib 7.

When locked, the tourniquet 1 is folded in the longitudinal direction to shorten its length, and then the folded part is placed in the crimping cover 6. The crimping cover 6 and the crimping surface are closed to let the rib 7 press against the folded part of the tourniquet 1, so the tourniquet 1 is set in tight state.

When unlocked, the crimping cover 6 and the crimping surface are opened to, and the rib 7 loosens the folded part of the tourniquet 1 so that the folded part can be extended, and the tourniquet 1 is set in loose state.

In order to further improve that the crimping cover 6 and the crimping surface can well compress the tourniquet 1, the groove 8 is provided at the position opposite to the rib 7, and the rib 7 presses the folded part of the tourniquet 1 into the groove 8 to fix. So, the folded part of the tourniquet 1 is further bent when the tourniquet 1 is in tight state, which increases the friction between the tourniquet 1 at the folded position and improves the stress when the tourniquet 1 is tightened, thereby more effectively ensuring tourniquet 1 in tight state to avoid loose.

The present invention also provides a specific embodiment of the structure between the crimping cover 6 and the crimping surface of the locking structure 2. Specifically, one end of the crimping cover 6 is connected with the unlock device 3 by a shaft, so the crimping cover 6 can be flipped to the unlock device 3 through the shaft to realize closing and opening. In order to realize locking in the closed state, the other end opposite to the shaft of the crimping cover 6 is buckled with the lock body 5 of the unlock device 3 through the lock buckle 4. When locked, the folded part of the tourniquet 1 is placed on the crimping surface, then the crimping cover 6 is rotated by the shaft and closed, and finally the lock buckle 4 is buckled with the lock body 5 to maintain the locked state.

### Embodiment 3

The lock structure 2 and the unlock device 3 are winding structures. Specifically, a winding shaft is used to fix the tourniquet 1 on. More specifically, the winding shaft can be set as a rotate fork. The winding shaft is connected with the drive part for driving the winding shaft to rotate.

When locked, the drive part drives the rotate fork to rotate, and the tourniquet 1 is wound on the rotate fork, so that the length of the tourniquet 1 is shortened, that is, in tight state.

When unlocked, the drive part reversely drives the rotating fork or disconnects with the rotate fork, so that the tourniquet 1 is unwound, that is, in loose state.

In the embodiment 1 and embodiment 2, the locking and unlocking between the lock buckle 4 and the lock body 5 is provided. Refer to FIG. 2, specifically, the lock buckle 4 and the lock body 5 are a lock hole and a bolt. The bolt is locked by the lock hole when it is inserted into the lock hole. The movement of the lock buckle 4 and the lock body 5 intersect to achieve locking and unlocking. The drive part that drives the locking buckle 4 and the lock body 5 can also be realized by different structures. The present invention provides the following structure for reference. One is using a motor. The shaft of the motor is equipped with an eccentric wheel. The end of the lock body 5 is matched with the eccentric wheel. When the motor rotates, the eccentric wheel rotates together. The eccentric wheel contacts with the lock body 5 and lift the lock body 5 up. Then the lock body 5 is reset by the spring, thereby realizing the unlocking and locking of the lock hole and the bolt. The other one is using a solenoid. The movable part of the solenoid is provided with a cone corresponding to the end of the lock body 5. The solenoid pulls the cone to move and lift the lock body 5. Then the lock body 5 is reset by the spring, thereby realizing the unlocking and locking of the lock hole and the bolt.

In order to separate the lock structure 2 from the unlock device 3 in time when unlocked, the unlock device 3 is provided with an ejection structure 10 along the insertion direction of the lock buckle 4, which at least includes a spring. The locking buckle 4 compresses the spring when inserted, and the spring will eject lock the buckle 4 when unlocked.

To avoid unexpected situations happens, such as battery is low, which may cause the lock buckle 4 and the lock body 5 to fail unlocking. The inventor also provided the unlock button 9 on the lock body 5. The unlock button 9 is connected to the lock hole. When pressed manually the unlock button 9, it moves the lock hole to release the bolt to unlock.

In order to realize the automatic unlocking of the unlock device, the control circuit set a control flow. A simpler one is to use a timing method, that is, when the tourniquet 1 is tightened, the control circuit automatically proceeds timing. When the time is up, the control circuit outputs a control signal to the drive part and drives the unlock device 3 to unlock. The timer of the control circuit can be triggered by the activate button 11. The activate button 11 is arranged on the surface where it is contact with the body. When the unlock device 3 is bound to the body by the tourniquet 1, the activate button 11 is pressed by the body to generate a signal and send to the control circuit for starting the timer to work. Furthermore, in order to notify relevant person in time to handle, the control circuit is also equipped with alarm devices, such as horns, lights, etc.

1. Automatic unlock device for tourniquet, wherein includes a lock structure for setting the tourniquet in tight state, an unlock device for unlocking and setting the tourniquet in loose state, and a control circuit for controlling the unlock device; the unlock device is connected to the lock structure, the drive part of the unlock device connects to the control circuit, and the drive part drives the unlock device to lock to or loose from the lock structure.

2. The automatic unlock device for tourniquet of claim 1, wherein the lock structure is a lock buckle arranged in the tourniquet; while locked, the lock buckle is buckled with a lock body in the unlock device to set the tourniquet in tight state; while unlocked, the lock buckle loose from the lock body to set the tourniquet in loose state; the lock body connects to the drive part of the unlock device.

3. The automatic unlock device for tourniquet of claim 1, wherein the lock structure is a crimping structure provided on the unlock device, which includes a crimping cover that is configured to relatively open/close with the crimping surface of the unlock device; a rib is provided on the crimping surface or the inner surface of the crimping cover; while locked, the tourniquet is folded along the longitudinal direction and placed in the crimping cover; the crimping cover and the crimping surface are closed to let the rib press against the folded part of the tourniquet, so the tourniquet is set in tight state; while unlocked, the crimping cover and the crimping surface are opened to let the rib loosen the folded part of the tourniquet, and the tourniquet is set in loose state.

4. The automatic unlock device for tourniquet of claim 3, wherein a groove is provided at a position opposite to the rib, and the rib presses the folded part of the tourniquet into the groove to fix.

5. The automatic unlock device for tourniquet of claim 3, wherein one end of the crimping cover is connected with the unlock device through a shaft, and the other end is buckled with the lock body of the unlock device by the lock buckle.

6. The automatic unlock device for tourniquet of claim 3, wherein the lock structure and the unlock device are winding structures, which include a winding shaft for the tourniquet being connected with; and the winding shaft is connected with the drive part; while locked, the drive part drives the winding shaft to wind the tourniquet on it, so the tourniquet is set in tight state; while unlocked, the drive part drives the winding shaft in the reverse direction or disconnects with the winding shaft to release the tourniquet for setting it in loose state.

7. The automatic unlock device for tourniquet of claim 3, wherein a lock hole and a bolt are respectively provided with the lock buckle and the lock body, and the bolt is locked by the lock hole after being inserted into the lock hole; the movement of the lock buckle and the lock body intersects.

8. The automatic unlock device for tourniquet of claim 7, wherein the lock body is provided with a unlock button, the unlock button is connected to the lock hole, and the unlock button is pressed to move the lock hole to unlock the bolt.

9. The automatic unlock device for tourniquet of claims 2, wherein the unlock device is provided with an ejection structure along the insertion direction of the lock buckle, which at least includes a spring; the lock buckle compresses the spring when inserted, and the spring ejects the lock buckle when unlocked.

10. The automatic unlock device for tourniquet of claims 2, wherein the drive part is a solenoid, and the end of the lock body is matched with the movable part of the solenoid.

11. The automatic unlock device for tourniquet of claims 2, wherein the drive part is a motor, the shaft of the motor is provided with an eccentric wheel, and the end of the lock body is matched with the eccentric wheel.

12. The automatic unlock device for tourniquet of claim 1, wherein a activate button for setting the timing of unlocking is provided, and the activate button is connected to the control circuit.

13. The automatic unlock device for tourniquet of claim 12, wherein the activate button is arranged on the surface of the unlock device where is contact with the body.

14. The automatic unlock device for tourniquet of claim 1, wherein an alarm device is provided in the control circuit.
